(19) United States
(12) Patent Application Publication
(10) Pub. No.: US 2005/0124886 A1
(43) Pub. Date: Jun. 9, 2005

(54) System and method for generating ultrasound images having variable spatial compounding

(75) Inventors: Ann O'Donnell, Seattle, WA (US); James Jago, Seattle, WA (US)

Correspondence Address: ATL ULTRASOUND, P.O. BOX 3003, 22100 BOTHELL EVERETT HIGHWAY, BOTHELL, WA, 98041-3003, US

(73) Assignee: Koninklijke Philips Electronics N.V.

(21) Appl. No.: 10/980,567

(22) Filed: Nov. 2, 2004

Related U.S. Application Data
(60) Provisional application No. 60/524,302, filed on Nov. 21, 2003.

(51) Int. Cl. A61B 8/00

(52) U.S. Cl. 600/443

(57) ABSTRACT

An ultrasound diagnostic imaging system and method produces spatially compounded images by combining component image frames acquired from different look directions. Different regions of the spatially compounded images are formed by different numbers of overlapping component frames. As a result, the degree of spatial compounding varies in these regions. The image frames in the regions are spatially filtered, temporally filtered or frequency compounded in a pattern that offsets the spatial variation in spatial compounding due to the different number of overlapping component frames in various regions of the image. As a result, the variations in spatial compounding are compensated for to provide an ultrasound image with more uniform speckle, noise, and temporal characteristics.

This invention claims the benefit of Provisional U.S. Patent Application Ser. No. 60/524,302, filed Nov. 21, 2003.

This invention relates to ultrasound diagnostic imaging systems and methods, and, more particularly, to ultrasound diagnostic imaging systems and method that produce spatially compounded images.

Spatial compounding is an imaging technique in which a number of ultrasound image frames of a target are obtained from multiple vantage points or angles. The image frames are then combined to produce a spatially compounded image by combining the data received from corresponding points in each of the image frames. Examples of spatial compounding may be found in U.S. Pat. Nos. 6,129,599 and 6,224,552, which are incorporated herein by reference. Real time spatial compound imaging is performed by rapidly acquiring a series of partially overlapping component image frames (i.e., typically greater than 10 image frames/second) from substantially independent spatial directions, utilizing an array transducer to implement electronic beam steering and/or electronic translation of the component frames. The component frames are combined by summation, averaging, peak detection, or other combinational means to produce a compound image. The acquisition sequence and formation of compound images are repeated continuously at a rate limited by the acquisition frame rate, that is, the time required to acquire the full complement of scanlines over the selected width and depth of imaging.

A spatially compounded image typically shows lower noise and speckle, and better specular reflector delineation, than conventional ultrasound images from a single viewpoint. Noise and speckle are reduced (i.e. speckle signal to noise ratio is improved) by the square root of N in a compound image with N component frames, provided that the component frames used to create the compound image are substantially independent and are averaged. Several criteria can be used to determine the degree of independence of the component frames (see, e.g., O'Donnell et al. in IEEE Trans. UFFC v.35, no.4, pp 470-76 (1988)). In practice, for spatial compound imaging with a steered linear array, this implies a minimum steering angle between component frames. This minimum angle is typically on the order of several degrees.

The second manner in which spatial compound scanning improves image quality is by improving the appearance of specular interfaces. For example, a curved bone-soft tissue interface produces a strong echo when the ultrasound beam is exactly perpendicular to the interface, and a very weak echo when the beam is only a few degrees off perpendicular. These interfaces are often curved so that, with conventional scanning, only a small portion of the interface is visible. Spatial compound scanning acquires views of the interface from many different angles, making the curved interface visible and continuous over a larger field of view. Greater angular diversity generally improves the continuity of specular targets. However, the angular diversity available is limited by the acceptance angle of the transducer array elements. The acceptance angle depends on the transducer array element pitch, frequency, and construction methods.

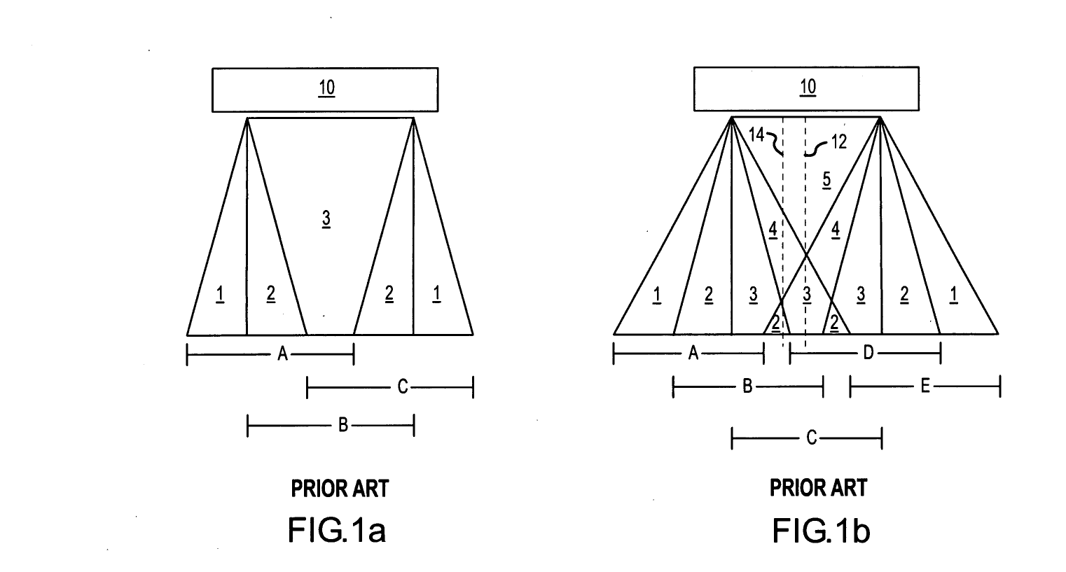

One of the problems that can arise when image frames from a plurality of look directions are acquired by a transducer is that all points in the ultimate compound image may not be created by data from the same number of image frames. Generally points in the central near field of the image will be formed from the greatest number of acquired image frames, while points at the lateral extremes and greater depths of the image are formed using data from fewer image frames. For example, as illustrated in FIG. 1a, a linear array transducer 10 scans three partially overlapping steered linear component image frames A-C. The transducer 10 steers the image frame A to the left, the image from C to the right, and the image frame B is not steered to either side. The degree of overlap of the component image frames A-C is different in various regions, and is designated by the underlined numerals in FIG. 1A. All three of the image frames A-C overlap in the region 3 beneath the center of the transducer 10, but only two image frames A,B and B,C overlap in the regions 2 to the left and right, respectively, of the center region. In the regions 1 beneath the edges of the transducer 10, there is no overlap in any of the image frames A-C. As a result, an ultrasound image obtained using the transducer 10 can have a fair degree of spatial compounding in the center region 3, but less spatial compounding in the regions 2 to the sides, and no spatial compounding in the regions 1 at the edges. The quality of the resulting image that can be obtained by spatial compounding will thus vary from a maximum quality at the center of the image and a lesser quality toward the sides of the image.

FIG. 1b shows the linear array transducer 10 scanning five component image frames A, B, C, D and E, with the number of image frames overlapping designated by the numerals 1-5. As in the image frames A-C of FIG. 1a, the number of overlapping image frames, and hence the degree of spatial compounding, varies from one side of the transducer 10 to the other. However, the number of overlapping image frames, and hence the degree of spatial compounding, also varies with depth. For example, the number of overlapping image frames along the line 12 varies from 5 adjacent the transducer 10, to 4 away from the transducer 10 and then finally to 3. Similarly, the number of overlapping image frames along the line 14 varies from 5 adjacent the transducer 10, to 4 and then 2 away from the transducer 10. The degree of noise and speckle reduction and the quality of specular reflector delineation that can be achieved with spatial compounding therefore varies with both width and depth, and is higher toward the center of the transducer and at shallower depths than it is toward the ends of the transducer and at greater depths.

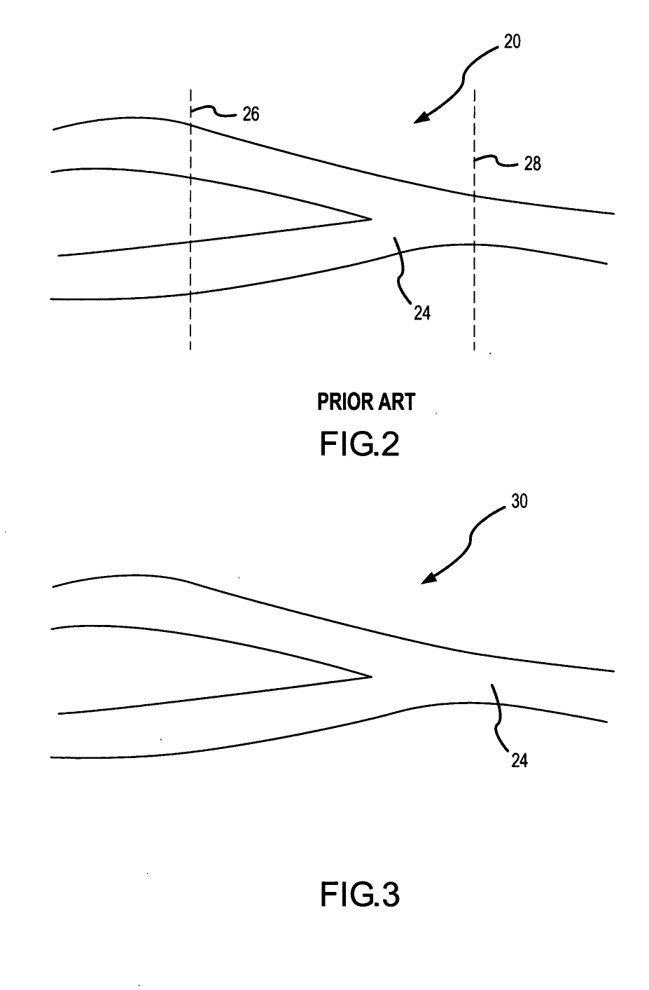

An example of a spatially compounded image that exhibits the problems described with reference to FIGS. 1A and 1B is represented in FIG. 2. FIG. 2 figuratively illustrates a B mode image 20 of a blood vessel 24 taken through a plane at the center of the vessel, which was obtained using spatial compounding. In FIG. 2 the speckling of the image 20 is greater at the edges of the image 20. This is because the amount of spatial compounding (i.e., the number of look directions from which samples are acquired and combined) is less on the lateral wings of the image outside the central area bounded by dashed lines 26,28.

One conventional means for providing a uniform image despite the above-described variations in spatial compounding is to crop the image to remove the portions in which the degree of spatial compounding is inadequate. For example, the image could be cropped beyond the lines 26, 28 as shown in FIG. 2. The resulting image would not include the lateral wing portions having the increased speckle, and would therefore be more uniform in speckle appearance. While this approach does improve the quality of the image, it can waste much of the useful information that is would otherwise be present in the image.

Another problem that is present in an image such as that of FIG. 2 is that the lateral wings result from one or a few number of temporally spaced component images, whereas the central portion of the image results from a greater number of temporally more frequent component images. This means that the central area of the image is updated more frequently in the live image sequence than are the lateral wings of the image. This regional variability of the updating of the image content is visually distracting to the user and detracts from a uniform image appearance. Accordingly it is desirable to reduce or eliminate this updating disparity of the image.

There is therefore a need for a system and method for generating spatially compounded images that compensates for variations in the degree of spatial compounding and updating disparity at different locations in the images yet allows the entire areas of the images to be used.

A method and system for generating spatially compounded ultrasound images includes an array transducer and beamformer for acquiring a plurality of ultrasound image frames from a zone of interest. The image frames are acquired at a plurality of respective look angles so that the number of image frames overlapping in different regions of the zone of interest varies. A processor processes the image frames to provide data corresponding to a spatially compounded image in which the degree of spatial compounding in each region varies. In particular, the degree of spatial compounding varies as a function of the number of overlapping image frames that are combined to form the spatially compounded image in the region. The processor also processes the image frames to compensate for the variations in the degree of spatial compounding in each region, such as by temporal processing, spatial processing, frequency compounding or by some other means. As a result, variations in the noise and speckle and temporal updating resulting from the spatial compounding variations are minimized. The spatially compounded ultrasound image is then generated from the image frames processed by the processor.

FIGS. 1a and 1b are schematic drawings illustrating the manner in which image frames used to form spatially compounded images overlap to different degrees in different regions beneath a transducer.

FIG. 2 is a schematic drawing of a B mode ultrasound image obtained using conventional spatial compound processing.

FIG. 3 is a schematic drawing of a B mode ultrasound image obtained using spatial compound processing according to one embodiment of the invention.

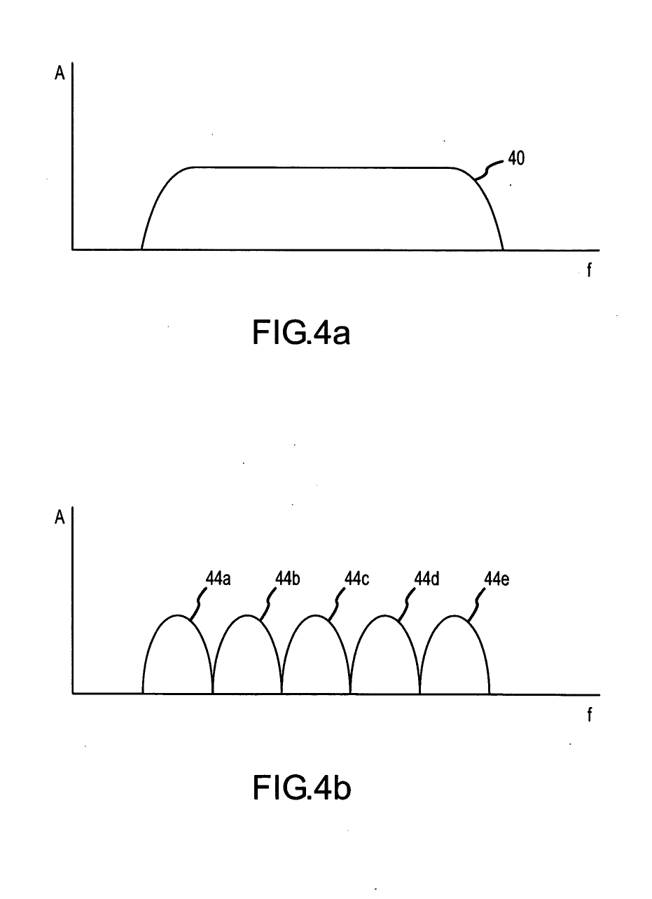

FIGS. 4a and 4b are a graph showing the frequency spectrum of ultrasound reflections and a graph showing the manner in which the frequency spectrum is divided into frequency bands for purposes of frequency compounding to compensate for variations in spatial compounding.

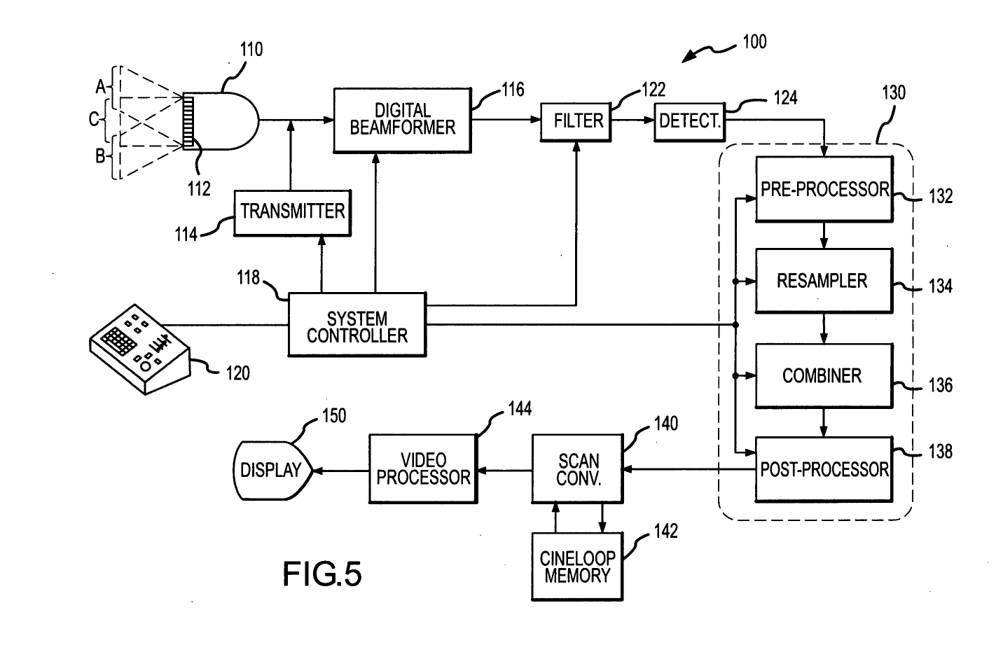

FIG. 5 is a block diagram of an ultrasound imaging system for generating spatially compounded ultrasound images in which variations in spatial compounding are compensated for by various means according to one embodiment of the invention.

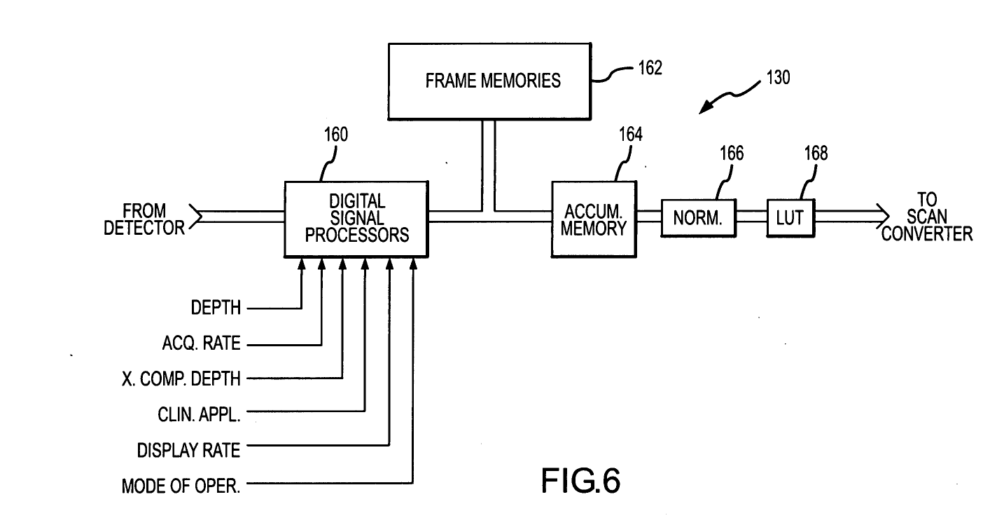

FIG. 6 is a block diagram of a spatial compounding processor used in the ultrasound imaging system of FIG. 5.

A system and method according to various embodiments of the invention makes spatially compounded images more uniform in appearance by providing additional processing in areas of the image that have been spatially compounded to a lesser degree. This additional processing is preferably at the edges of an image in which the degree of spatial compounding is inherently diminished. In one embodiment of the invention, the temporal persistence of an image is increased in areas that are toward the edges of the image compared to areas toward the center of the image. The temporal persistence can be increased by combining image frames that have been acquired at different times to generate the area of the image near its edges. For example, with reference to FIG. 1b, the areas of the image corresponding to the regions 1 in which there are no overlapping image frames are obtained by combining 5 image frames obtained on 5 successive scans. The areas of the image corresponding to the regions 2 in which there are 2 overlapping image frames are obtained by combining image frames obtained on 4 successive scans. Similarly, the areas of the image corresponding to the regions 3 in which there are 3 overlapping image frames are obtained by combining image frames obtained on 3 successive scans, the areas of the image corresponding to the regions 4 are obtained by combining image frames obtained on 2 successive scans, and the area of the image corresponding to the region 5 is obtained by the image frames for only the current scan. The noise and speckle in each image frame is random in nature. Therefore, combining multiple image frames obtained at different time reduces the noise and speckle that is present in any one image frame and produces the sense of image updating across the image. The noise and speckle are therefore reduced in a manner that is similar to the reduction in noise and speckle resulting from spatially compounding the image frames, and the temporal disparity across the image is also reduced.

FIG. 3 represents a B mode image 30 of the blood vessel 24 also taken through the center of the blood vessel, which was obtained using spatial compounding and temporal averaging to compensate for variations in the amount of spatial compounding. In FIG. 3 the temporal updating of the image 30 appears more uniform across the width of the image 30 and no longer appears more static toward the edges of the image 20 as represented in FIG. 2. The speckling of the image 30 is also more uniform across the width of the image 30 compared to the image 20 of FIG. 2.

In another embodiment of the invention, the variations in spatial compounding are compensated for by spatial filtering. Specifically, the degree of spatial filtering is greater toward the edges of an image where there is little or no spatial compounding. Little or no spatial filtering is provided toward the center of the image where there is a substantial amount of spatial compounding. Various types of spatial filtering are well-known in the art, including simple smoothing of image pixels, median filters and adaptive filters. A filter which can produce satisfactory results in many applications is a symmetrical spatial filter with the size or weighting of the filter kernel matching the degree of filtering desired.

Still another embodiment of the invention uses frequency compounding to compensate for variations in spatial compounding in an image. FIG. 4a shows the frequency spectrum 40 of ultrasound reflections from tissues beneath an ultrasound transducer (not shown in FIG. 4a). As shown in FIG. 4b, the frequency spectrum 40 can be divided into several bands 44a-e by conventional means, such as bandpass filtering, and the number of bands used to create each area of an image is selected to compensate for the variations in spatial compounding. More specifically, the frequencies in each ultrasound echo are split into the bands 44a-e and separately detected, and the separately detected signals, each with a different speckle characteristic, are recombined as described in greater detail in U.S. Pat. No. 4,561,019 (Lizzi et al.) The speckle and noise are different for each band 44a-e, so that the areas of the image obtained by processing reflections in multiple frequency bands 44a-e has the effect of averaging the speckle present in any one band over all of the bands 44a-e used to form an area of the image. For example, with reference to FIG. 1b, the areas of the image corresponding to the regions 1 in which there are no overlapping image frames are obtained by processing reflections in all 5 frequency bands 44a-e, the areas corresponding to the regions 2 are obtained by processing reflections from the passband 40 divided into only 4 frequency bands, the areas corresponding to the regions 3 are obtained by processing reflections from the passband 40 divided into only 3 frequency bands, the areas corresponding to the regions 4 are obtained by processing reflections from the passband 40 divided into only 2 frequency bands 44b-c, and the areas corresponding to the region 5 is obtained by processing reflections in the undivided passband 40. Thus, speckle reduction due to frequency compounding is done in inverse proportion to that achieved by spatial compounding in different areas of the image.

One embodiment of an ultrasound diagnostic imaging system 100 that may be used to implement the various embodiments of the invention is shown in FIG. 5. The imaging system 100 includes a scanhead 110 having an array transducer 112 that transmits beams at different angles over an image field denoted by dashed rectangle and parallelograms. Three groups of scanlines are indicated in the drawing, labeled A, B, and C, with each group being steered at a different angle relative to the scanhead 110. The transmission of the beams is controlled by a transmitter 114, which controls the phasing and time of actuation of each of the elements of the array transducer 112 so each beam is transmitted from a predetermined origin along the array and at a predetermined angle. The echoes returned from along each scanline are received by the elements of the array, digitized as by analog-to-digital conversion, and coupled to a digital beamformer 116. The digital beamformer 116 delays and sums the echoes from the array elements of the transducer 112 to form a sequence of focused, coherent digital echo samples along each scanline. The sequence of samples are used to form respective image frames corresponding to the beam formed by the beamformer 116. The transmitter 114 and beamformer 116 are operated under control of a system controller 118, which in turn is responsive to the settings of controls on a user interface 120 operated by the user of the ultrasound system 100. The system controller 118 controls the transmitter 114 to transmit the desired number of scanline groups at the desired angles, transmit energies and frequencies. The system controller 118 also controls the digital beamformer 116 to properly delay and combine the received echo signals for the apertures and image depths used.

The scanline echo signals are filtered by a programmable digital filter 122, which defines the band of frequencies of interest. When imaging harmonic contrast agents or performing tissue harmonic imaging, the passband of the filter 122 is set to pass harmonics of the transmit band. The filtered signals are then detected by a detector 124. In one embodiment of the invention, the filter 122 and detector 124 include multiple filters and detectors so that the received signals may be separated into multiple passbands as shown in FIG. 4b, individually detected and recombined for frequency compounding to compensate for variations in the degree of spatial compounding, as explained above. For B mode imaging, the detector 124 performs amplitude detection of the echo signal envelope. For Doppler imaging, ensembles of echoes are assembled for each point in the image and are Doppler processed to estimate the Doppler shift or Doppler power intensity.

In accordance with various embodiments of the present invention, the digital echo signals are processed by spatial compounding in a spatial compounding processor 130. The processor 130 also performs additional processing to compensate for variations in the degree of spatial compounding in different regions of tissues or fluids beneath the scanhead 110. This additional processing can be temporal processing, spatial processing or frequency compounding, as described above, or some other type of processing that can compensate for variations in the degree of spatial compounding. The digital echo signals are initially pre-processed by a preprocessor 132. The preprocessor 132 can preweight the signal samples if desired with a weighting factor. The samples can be preweighted with a weighting factor that is a function of the number of component frames used to form a particular image. The pre-processed signal samples may then undergo a resampling in a resampler 134. The resampler 134 can spatially realign the estimates of one component frame or to the pixels of the display space.

After the pre-processed signal samples have been resampled, the image frames are compounded by a combiner 136 as explained above. As also previously explained, the number of image frames compounded by the combiner 136 will vary depending upon the number of beams overlapping in each location. The compounding accomplished by the combiner 136 may comprise summation, averaging, peak detection, or other combinational means. The samples being combined may also be weighted prior to combining in this step of the process. Finally, post-processing is performed by a post-processor 138. The post-processor 138 normalizes the combined values to a display range of values, and it also performs temporal or spatial processing to compensate for variations in the degree of spatial compounding provided by the combiner 136. Post-processing can be most easily implemented by look-up tables, and can simultaneously perform compression and mapping of the range of compounded values to a range of values suitable for display of the compounded image.

The compounding process may be performed in estimate data space or in display pixel space. In a preferred embodiment scan conversion is done following the compounding process by a scan converter 140. The compound images may be stored in a Cineloop memory 142 in either estimate or display pixel form. If stored in estimate form, the images may be scan converted when replayed from the Cineloop memory 142 for display. The scan converter 140 and Cineloop memory 142 may also be used to render three dimensional presentations of the spatially compounded images as described in U.S. Pat. Nos. 5,485,842 and 5,860,924, which are incorporated herein by reference. Following scan conversion, the spatially compounded images are processed for display by a video processor 144 and displayed on an image display 150.

FIG. 6 illustrates one embodiment of the spatial compounding processor 130 of FIG. 5. The processor 130 is preferably implemented by one or more digital signal processors 160, which process the image data in various ways. The digital signal processors 160 can weight the received image data and can resample the image data to spatially align pixels from frame to frame, for instance. The digital signal processors 160 direct the processed image frames to a plurality of frame memories 162, which buffer the individual image frames. The number of image frames capable of being stored by the frame memories 162 is preferably at least equal to the maximum number of image frames to be compounded, such as sixteen frames. In accordance with the various embodiments of the present invention, the digital signal processors 160 are responsive to control parameters including data identifying the degree of spatial compounding in each region, for compensating for variations in the degree of spatial compounding by temporal processing, spatial processing, frequency compounding or some other means. The digital signal processors 160 select component frames stored in the frame memories 162 for assembly as a compound image in accumulator memory 164. The compounded image formed in the accumulator memory 164 is weighted or mapped by a normalization circuit 166, then compressed to the desired number of display bits and, if desired, remapped by a lookup table (LUT) 168. The fully processed compounded image is then transmitted to the scan converter 140 (FIG. 5) for formatting and display.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method for generating spatially compounded ultrasound images, comprising:
acquiring a plurality of ultrasound image frames of a zone of interest, the image frames being acquired at a plurality of respective look angles in a manner in which the number of image frames overlapping in different regions of the zone of interest varies;
processing the image frames to provide data corresponding to a spatially compound image in which the degree of spatial compounding in each region varies as a function of the number of overlapping image frames combined to form the spatially compounded image in the region;
processing the image frames to compensate for the variations in the degree of spatial compounding in each region to reduce variations in the noise, speckle, and temporal appearance resulting from the spatial compounding variations; and
generating a spatially compounded ultrasound image from the processed image frames.

2. The method of claim 1 wherein the act of processing the image frames to compensate for the variations in the degree of spatial compounding in each region comprises differently temporally processing lateral portions of the image frames as compared to a central portion.

3. The method of claim 2 wherein the act of temporally processing the image frames comprises combining a number of image frames acquired at respective times, the number of image frames acquired at respective times that are combined for each region of the image being inversely related to number to the number of image frames acquired at respective look angles for spatial compounding in corresponding regions of the zone of interest.

4. The method of claim 1 wherein the act of processing the image frames to compensate for the variations in the degree of spatial compounding in each region comprises differently spatially processing lateral portions of the image frames as compared to a central portion.

5. The method of claim 1 wherein the act of processing the image frames to compensate for the variations in the degree of spatial compounding in each region comprises frequency compounding the image frames.

6. The method of claim 5 wherein the act of frequency compounding the image frames comprises dividing ultrasound reflections into a plurality of frequency bands and using the ultrasound reflections in the frequency bands to generate the image frames, the number of frequency bands used to generate each region of the image inversely corresponding in number to the number of image frames acquired at respective look angles for spatial compounding in corresponding regions of the zone of interest.

7. A method for generating a spatially compensated ultrasound image, comprising:
transmitting a plurality of beams of ultrasound into tissues or fluid of interest;
receiving ultrasound echoes resulting from the transmitted ultrasound;
beamforming the received ultrasound echoes to obtain signals corresponding to image frames, the received ultrasound echoes being beamformed to form a plurality of image frames extending in a plurality of respective directions to insonify the tissues or fluid of interest from one side, through a center region to an opposite side of the tissues or fluid of interest; and
generating each of a plurality of areas of the spatially compensated ultrasound image by combining signals from each image frame that insonifies a respective region of the tissues or fluid of interest thereby spatially compounding the image, the spatially compensated ultrasound image being generated by processing signals from ultrasound reflections at the edges of the tissues or fluid of interest to a greater extent by means other than spatial compounding than ultrasound reflections at the center region of the tissues or fluid of interest to compensate for the variations in the degree of spatial compounding in each the tissues or fluid of interest.

8. The method of claim 7 wherein the act of processing signals from ultrasound reflections at the edges of the tissues or fluid of interest to a greater extent than ultrasound reflections at the center region of the tissues or fluid of interest comprises temporally processing the signals from ultrasound reflections at the edges of the tissues or fluid of interest to a greater extent than signals from ultrasound reflections at the center region of the tissues or fluid of interest.

9. The method of claim 7 wherein the act of processing signals from ultrasound reflections at the edges of the tissues or fluid of interest to a greater extent than ultrasound reflections at the center region of the tissues or fluid of interest comprises spatially processing the signals from ultrasound reflections at the edges of the tissues or fluid of interest to a greater extent than signals from ultrasound reflections at the center region of the tissues or fluid of interest.

10. The method of claim 7 wherein the act of processing signals from ultrasound reflections at the edges of the tissues or fluid of interest to a greater extent than ultrasound reflections at the center region of the tissues or fluid of interest comprises frequency compounding the signals from ultrasound reflections at the edges of the tissues or fluid of interest to a greater extent than signals from ultrasound reflections at the center region of the tissues or fluid of interest.

11. An ultrasound diagnostic imaging system for generating a spatially compounded ultrasound image of blood or tissue in a region of interest, the system comprising:
a scanhead having an array transducer for scanning the region of interest;
a transmitter selectively applying transmit signals to the transducer;
a beamformer coupled to receive echo signals from the transducer and to combine the received echo signals into output signals corresponding to respective image frames that are steered in a variety of directions;
a processor coupled to the beamformer, the processor being operable to spatially compound the image frames in a manner that causes the degree of spatial compounding to vary as a function of respective locations in the region of interest from which the echo signals are received, the processor further being operable to process the image frames to compensate for the variations in the degree of spatial compounding in each region of interest; and
a display subsystem coupled to the processor for displaying the spatially compounded ultrasound image from the spatially compound the image frames after being processed to compensate for the variations in the degree of spatial compounding.

12. The ultrasound diagnostic imaging system of claim 11 wherein the processor is operable to process the image frames to compensate for the variations in the degree of spatial compounding in each region of interest by temporally processing the image frames.

13. The ultrasound diagnostic imaging system of claim 11 wherein the processor is operable to process the image frames to compensate for the variations in the degree of spatial compounding in each region of interest by spatially processing the image frames.

14. The ultrasound diagnostic imaging system of claim 11 wherein the processor is operable to process the image frames to compensate for the variations in the degree of spatial compounding in each region of interest by frequency compounding the image frames.

15. The ultrasound diagnostic imaging system of claim 11 wherein the processor comprises:
a pre-processor having an input that is coupled to an output from the beamformer, the pre-processor being operable to process sample of signals from the beamformer;
a resampler having an input that is coupled to an output from the pre-processor, the resampler being operable to spatially realign the samples;
a combiner having an input that is coupled to an output from the resampler, the combiner being operable to perform spatial compounding of the spatially realigned samples; and
a post-processor having an input that is coupled to an output from the combiner, the post-processor being operable to process signals from the combiner to compensate for variations in the degree of spatial compounding performed by the combiner.

16. The ultrasound imaging system of claim 11 wherein the display subsystem comprises:
a scan converter having an input coupled to an output of the processor;
a video processor having an input coupled to an output of the scan converter; and
a display unit having an input coupled to an output of the video processor.

17. The ultrasound diagnostic imaging system of claim 11 wherein the processor comprises:
a plurality of digital signal processors having an input coupled to the beamformer, the digital signals processors being operable to generate data corresponding to spatially compounded image frames and to compensate for variations in the degree of spatial compounding at different locations in the region of interest;
a plurality of frame memories having respective inputs coupled to respective outputs of the digital signal processors, the frame memories being operable to store respective image frames; and
an accumulator memory storing a spatially compounded image frame created from a plurality of image frames stored in respective ones of the frame memories as selected by the digital signal processors.

18. The ultrasound diagnostic imaging system of claim 11 wherein the image frame correspond in number to the maximum number of image frames that are combined to generate the spatially compounded ultrasound image.

19. An ultrasound image corresponding to blood or tissues in a region of interest comprising a spatially compounded ultrasound image having variations in the degree of spatial compounding from one side of the image to another side of the image, the ultrasound image having substantially uniform speckle and/or temporal characteristics from the one side of the image to the another side of the image despite the variations in spatial compounding of the image.
